(19) United States
(12) Patent Application Publication
(10) Pub. No.: US 2023/0248964 A1
(43) Pub. Date: Aug. 10, 2023

(54) INTRAVAGINAL ELECTRICAL STIMULATOR DEVICE

(71) Applicant: NOVANEXT, LLC, Falls Church, VA (US)

(72) Inventor: Seyed Abbas Shobeiri, Falls Church, VA (US)

(73) Assignee: NOVANEXT, LLC, Falls Church, VA (US)

(21) Appl. No.: 18/102,847

(22) Filed: Jan. 30, 2023

Related U.S. Application Data
(60) Provisional application No. 63/307,226, filed on Feb. 7, 2022.

(51) Int. Cl. A61N 1/05; A61N 1/372; A61N 1/36

(52) U.S. Cl. CPC A61N 1/0524; A61N 1/36007; A61N 1/37247

(57) ABSTRACT

A device for muscle training and treating urinary incontinence including a body configured for insertion intravaginally for placement within the vagina, at least one sensor supported on the body for measuring intra-abdominal pressure and at least one electrode supported on the body and in electrical communication with the at least one sensor. The at least one electrode sends an electrical signal to pelvic muscles of a user to contract the pelvic muscles based on feedback from the sensor. An electric signal generator housed within the body sends the electrical signal to the electrode. The device can also have application for treating fecal incontinence and pelvic floor weakness.

### BACKGROUND OF THE INVENTION

This application claims priority from Provisional Application Serial no. 63/307,226, filed Feb. 7, 2022, the entire contents of which are incorporated herein by reference.

### Field of the Invention

This application is directed to a device for muscle training and/or treating urinary incontinence, fecal incontinence, and/or pelvic floor weakness, and more particularly, to an intravaginal device for providing electrical stimulation to contract pelvic muscles for muscle training and/or treating urinary incontinence, fecal incontinence and/or pelvic floor weakness.

### Background

It is estimated that there are over 40 million women in the United States and over 250 million women worldwide that suffer from female urinary incontinence (UI). Urinary incontinence increases in severity with age, leading to higher rates for older women.

Stress urinary incontinence is the involuntary leakage of urine. Leakage due to UI can be caused for example, by laughing, sneezing, coughing, and exercise/physical activities such as lifting or running. These activities cause an increase in intra-abdominal pressure causing bladder pressure. On average, there is a 6.5-year delay from the onset of symptoms until diagnosis and treatment due oftentimes to denial and embarrassment. UI may be associated with depression, psychological distress, and lost time from work.

There are several current methods for treating UI. One method involves physical therapy to strengthen and gain more control of the pelvic muscles. Another method is using an electrical stimulation device which is designed to cause contraction of the pelvic muscles and strengthening of these muscles. These stimulation devices typically provide a series of pulses via electrodes to the vaginal wall. However, such electrical stimulation devices are rarely used because they are inconvenient and burdensome. They require placement in the vagina several times a day to stimulate the muscles and require the woman to lay in bed for about twenty minutes. Further, these devices include a one-inch ring placed around the probe and a wire extending to an external battery pack. This increases discomfort and complicates use.

The need exists for a device that overcomes the deficiencies of the foregoing devices. Such device would advantageously be easy to use, not place an undue burden on the user and effectively and non-surgically train pelvic floor muscles and treat UI, (as well as fecal incontinence and/or pelvic floor weakness). It would further be advantageous if such device could be used in stationary as well as ambulatory positions of the user.

### SUMMARY OF THE INVENTION

The present invention overcomes the problems and deficiencies of the prior art. The present invention provides a smart electrical stimulator device for muscle training, e.g., pelvic floor training and pelvic floor weakness and/or treating urinary incontinence (UI) (and other treatments such as fecal incontinence and pelvic floor weakness). The device applies electrical energy to provide electrical stimulation to contract the pelvic muscles to limit leakage in response to increased abdominal pressure which causes increased bladder pressure or vaginal and pelvic floor axis change. Thus, the device of the present invention prevents urinary incontinence during acute activities that generate sudden intra-abdominal pressure changes. The device provides feedback from the abdominal pressure to pelvic floor muscles for the purpose of activation of the pelvic floor muscles and prevention of urinary incontinence and/or fecal incontinence.

The stimulator device of the present invention is inserted minimally invasively intravaginally for placement within the vagina. The device is lightweight, has a compact and streamlined design for ease of user insertion and user comfort, and can be used in stationary/supine conditions of the user as well as in ambulatory/active conditions of the user. The device is also preferably wireless, so it is not cumbersome to use and does not inhibit user mobility or user activity.

There are various device modes, such as selective, automatic, and biofeedback. The device can also be used continuously in a therapeutic mode or activated in response to pressure readings in a treatment mode. Each of these modes is discussed in detail below. Data storage capabilities in the device and/or in a remote device are also provided in some embodiments, discussed in detail below.

In accordance with one aspect of the present invention, an intravaginal stimulator device is provided comprising a) a body configured for insertion intravaginally for placement within the vagina, the body having a head section, a middle section, and a tail section; b) at least one sensor supported on the body for measuring intra-abdominal pressure or vaginal axis change; c) at least one electrode supported on the body and in electrical communication with the at least one sensor, the at least one electrode sending an electrical signal to the pelvic muscles of a user to contract the pelvic muscles based on feedback from the sensor; and d) an electric signal generator housed within the body for sending the electrical signal to the electrode. The device in some embodiments is configured for remote activation by an external control in wireless communication with the device, wherein the device has a selective mode and an automatic mode. In the selective mode, a signal is sent by the generator to the electrode(s) in response to measured abdominal pressure based on a selective activation to send electrical energy to the pelvic muscles of the user to contract the pelvic muscles. In the automatic mode, the signal is automatically sent to the electrode to send electrical energy to the pelvic muscles to contract the pelvic muscle in response to measured intraabdominal pressure by the sensor.

In some embodiments, the intensity of the electrical stimulation is adjustable by the user/patient.

In some embodiments, the device has a first sensor at the tail section and a second sensor at the head section. In some embodiments, a first sensor is on one side of the body and a second sensor is on an opposing side of the body.

In some embodiments, the at least one electrode comprises a first electrode on one side of a middle section of the body and a second electrode on an opposing side of the middle section of the body. In some embodiments, the at least one electrode and/or the at least one sensor is built into the body to provide a smooth outer surface of the device.

In some embodiments, the electrical signals are generated within the device by the generator within the body such that the device does not have an external connector and is fully contained within the vagina.

In some embodiments the device has an extraction ring.

In some embodiments, a signal is sent to the electrode only if intra-abdominal pressure exceeds a predetermined threshold. In some embodiments, a signal is sent to the electrode only if an increase in intra-abdominal pressure exceeds a predetermined amount. Thus, a comparative analysis is conducted.

In some embodiments, one or more of light detection, ultrasound or motion detection activates at least one electrode.

In some embodiments, the device is in wireless communication with a smart external electronic control, the external control having a graphic display to display one or more of a) the abdominal pressure; b) a surge in abdominal pressure; and/or c) the intensity of the electrical energy.

In some embodiments, data is collected and stored of parameters of use of the device of one or more of a) the abdominal pressure; b) a surge in abdominal pressure; c) the intensity of the electrical energy; and/or d) time period of electrical energy application. The data can be stored on one or both of the device itself and/or an external device. The external storage device could also be the device for controlling energy applications.

In some embodiments, the device is configured to be in communication with a Mobile App running on an external smartphone, which captures the pressure and motion gradient and provides for biofeedback, electrical stimulation, and pressured-triggered stimulation. In other embodiments, the device is configured to communicate with software running on a personal computer to provide for biofeedback and programming by the provider only. A provider is defined as a trained individual in device adjustment.

In accordance with another aspect of the present invention, an intravaginal stimulator device is provided comprising a) a body configured for insertion intravaginally for placement within the vagina, the body having a head section, a middle section and a tail section, the tail section having a transverse dimension less than a transversion dimension of the middle section; b) at least one sensor supported on the body for measuring intra-abdominal pressure; c) at least one electrode supported on the body and in electrical communication with the at least one sensor, the at least one electrode sending an electrical signal to pelvic muscles of a user to contract the pelvic muscles based on feedback from the sensor; and d) an electric signal generator housed within the body for sending the electrical signal to the electrode. The device is configured for use during a stationary and ambulatory/active condition of the user.

In accordance with another aspect of the present invention, a method for contracting pelvic muscles for treating e.g., urinary incontinence or vaginal axis exchange, is provided comprising intravaginally inserting a device having a) at least one sensor supported on the body for measuring intra-abdominal pressure, b) at least one electrode supported on the body and in electrical communication with the at least one sensor, and c) an electric signal generator housed within the body for sending the electrical signal to the electrode, the at least one electrode sending an electrical signal to pelvic muscles of a user to contract the pelvic muscles based on feedback from the sensor. The method can further include remotely activating the device via an external control in wireless communication with the device, wherein activating comprises either i) enabling a selective mode wherein the user remotely selectively sends a signal to the electrode to send electrical energy to contract the pelvic muscles of the user in response to measured intra-abdominal pressure by the sensor; or ii) enabling an automatic mode wherein the signal is automatically sent to the electrode to send electrical energy to contract the pelvic muscles of the user in response to measured intra-abdominal pressure by the sensor.

In the method of use, the device is preferably usable in stationary and active positions of the user.

In some embodiments, the method further includes storing data such as electrical stimulation intensity, time periods of electrical stimulation and/or measured intra-abdominal pressures.

### BRIEF DESCRIPTION OF THE DRAWINGS

So that those having ordinary skill in the art to which the subject invention appertains will more readily understand how to make and use the apparatus (device) disclosed herein, preferred embodiments thereof will be described in detail hereinbelow with reference to the drawings, wherein:

FIG. 1 illustrates the electrical stimulator device of the prior art positioned within the vagina for treating urinary incontinence (UI);

FIG. 2 is an anatomical view illustrating increased abdominal pressure and bladder pressure during physical activity;

FIG. 3 is a schematic left-side view of the electrical stimulator device of the present invention illustrating the left electrode and two sensors;

FIG. 4 is a schematic side view of the stimulator device of FIG. 3 shown positioned within the vagina for treatment of UI;

FIG. 5 is a schematic anterior view of the stimulator device of FIG. 3, showing the two sensors and the left and right electrodes;

FIG. 6 is a schematic head view of the stimulator device of FIG. 3 adjacent to a ruler to illustrate the dimensions;

FIG. 7 is a schematic tail view of the stimulator device of FIG. 3 adjacent a ruler to illustrate the dimensions;

FIG. 8A is a schematic view showing the stimulator device of FIG. 3 in a resting condition and the muscles under normal pressure;

FIG. 8B is a view similar to FIG. 8A showing the contraction of the pelvic muscles in response to the activation of the device; and

FIG. 9 is a top view of an alternate embodiment of the stimulator device of the present invention.

### DETAILED DESCRIPTION OF PREFERRED EMBODIMENTS

The present invention is directed to a smart electrical stimulator device for muscle training, e.g., pelvic floor training and/or treating urinary incontinence (UI). At the outset, it should be appreciated that the smart device disclosed herein can also be used for other treatments such as overactive bladder, fecal incontinence, pelvic floor weakness, etc. Also, the device of the present invention can be implanted in various regions of the body for various treatments via the application of electrical energy. The device is described in detail below for placement in the vagina to treat UI by way of example.

The stimulator device of the present invention is inserted minimally invasively intravaginally for placement within the vagina. The device senses intra-abdominal pressure and/or sudden intra-abdominal pressure increase and triggers electrodes to provide electrical stimulation to recruit the pelvic floor muscles, i.e., to contract the pelvic muscles. Leakage due to UI can be caused by laughing, sneezing, coughing, running water, or exercise/physical activities such as lifting or running which can cause increase in abdominal pressure. Electrical stimulation limits leakage from these triggers due to muscle contraction as described in detail below. In some embodiments, the device is used continuously for a predetermined amount of time and at a predetermined frequency as a therapeutic device to achieve longer term muscle contractions (referred to herein as a “continuous mode”). In other embodiments, the device is activated in response to certain intra-abdominal pressures, i.e., exceeding a predetermined threshold, to provide treatment (referred to herein as a “selective mode” or an “intermittent mode”). The device can also provide a “biofeedback mode” wherein the pressure measurement and number and duration of electrical pulses, as well as the frequency in embodiments where the frequency can vary during treatment, can be stored/recorded for analysis by the user and/or clinician and adjusted for the particular user. Such data can also be collected from devices used by different users/patients, and the data used and shared among clinicians for overall treatment evaluation and advancements. The data can be stored in memory in the device itself or transferred to and stored in another storage device, i.e., a remote device. Thus, the data can be stored for data acquisition to provide post-use evaluation. In some embodiments, the data can be downloaded from the device memory via a data transfer interface (e.g., USB). Data can be sent to a center for trending and optimization for future uses and clinical applications. As an alternative option, the device can be connected via Bluetooth/wireless on their iPad or other mobile devices for data transfer. In some embodiments, that data can be evaluated by the user to determine trends and inform future device use.

The device (probe) of the present invention is lightweight and ambulatory. In preferred embodiments it is soft and flexible/compliant, and can be washable. Its streamlined shape aids insertion and reduces discomfort to the user (also referred to herein as the patient). The device is also compact and can be, for example, less than about 8 centimeters (although other dimensions are also contemplated). It can have a weight of less than about 150 grams, although other weights are also contemplated. It can have automatic controls for treatment or, alternatively can be controlled by the user. Each of these uses are described in detail below. Further, being ambulatory, the user has full mobility during use. Thus, the device can be used with the patient in either supine or ambulatory positions. The device can remain in place and allow the patient to perform normal activities, including exercise. Thus, the smart vaginal electrical stimulator can advantageously be used in standing, bending, stooping, or running positions. The device is also preferably wireless, with no external connectors, which better enables activity during use.

The device of the present invention prevents urinary incontinence during acute activities that generate sudden intra-abdominal pressure changes. The device provides feedback from the abdominal pressure to pelvic floor muscles for the purpose of activation of the pelvic floor muscles and prevention of urinary incontinence (and/or fecal incontinence).

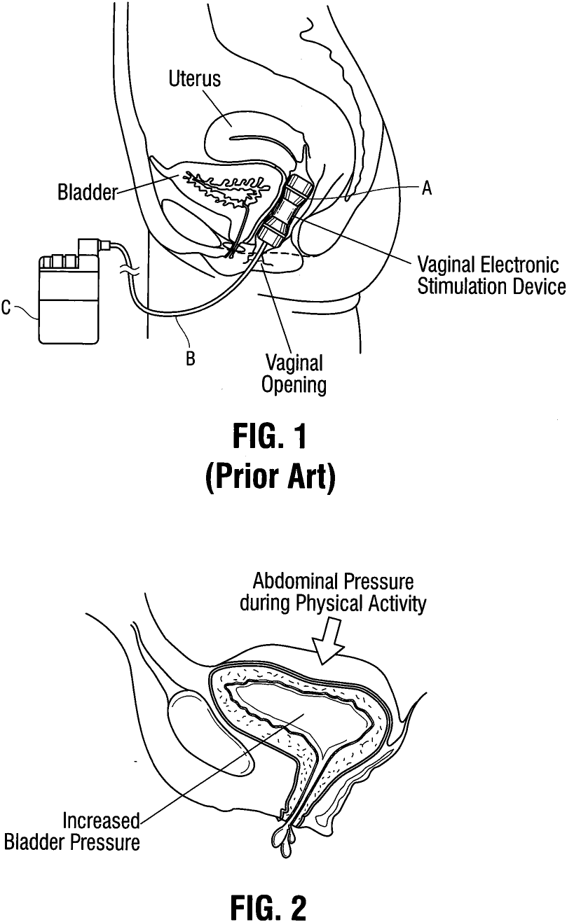

As shown in FIG. 1, the prior art device A is connected via connector B to power box C external of the user’s body. That is, a connector B, e.g., wires, extends from the device electrodes and out of the vagina to monitor C. Monitor C includes a signal generator to provide electrical signals to the electrodes for electrical stimulation. The external connector, with the attached external monitor, limits the wearer’s mobility and provides a more cumbersome system. In contrast, in preferred embodiments of the present invention, there are no external connectors or wires extending outside the vagina as the device is a self-contained all-in-one unit, generating electrical pulses or continuous waves from within the device, as well as data collection in some embodiments.

Note, as used herein, the term “proximal” or “head” refers to the portion or region of the device closer to the user, and the term “distal” or “tail” refers to the portion or region of the device further from the user. Thus, the device is inserted into the vagina “tail first.” The terms “device” and “apparatus” are used herein interchangeably.

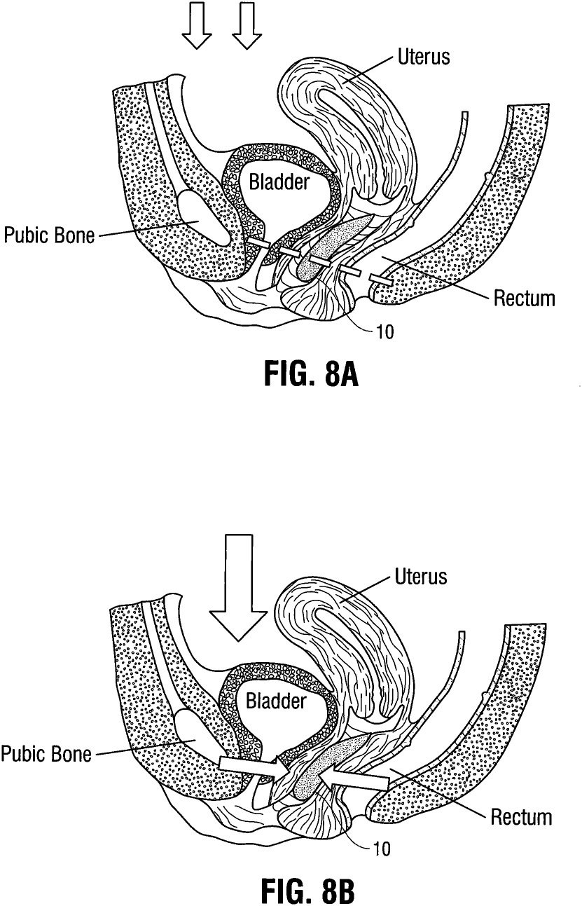

Referring now to the drawings and particular embodiments of the present disclosure, wherein like reference numerals identify similar structural features of the devices throughout the several views, the smart vaginal electrical stimulator device is designated generally by reference numeral 10 and is shown for insertion into the vagina to measure increased bladder pressure in response to increased abdominal pressure during physical activity (see anatomical representations in FIGS. 8A and 8B). With initial reference to FIG. 4, the device 10 (also referred to herein as probe 10) is positioned within the vagina, preferably touching (in contact/abutment with) the walls of the vagina to provide vaginal mucosal contact, but alternatively can be spaced therefrom. The device 10 has one or more sensors to sense/detect intraabdominal pressure and intraabdominal pressure increase, e.g., a sudden increase in pressure, which increases bladder pressure due to the triggers/causes enumerated above. That is, the sensors face the bladder and abdominal area and detect increased pressure, e.g., sudden increased pressure, which can be caused by laughing, sneezing, coughing, lifting, running, exercise, etc. The intraabdominal pressure change may be calculated or tabulated by motion movement sensors, ultrasound detection, pressure sensors or optical sensors of the axis of the vagina, bladder, rectum or levator ani muscles. The device 10 also has electrodes to provide electrical energy, which can be in the form of electrical pulses (pulse waves or longer continuous waves), which activate the pelvic muscles to close and prevent urine or fecal leakage or pelvic organ prolapse in response to sensed pressure detected by the sensors(s).

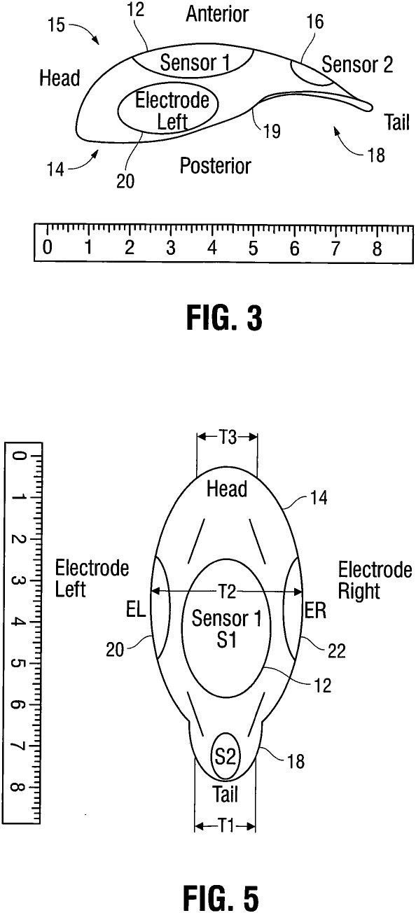

In the embodiment of FIGS. 3-7, the device 10 has two sensors supported on the device body (also referred to herein as the shell) to provide sidewall electrical contacts: sensor 12 positioned in the middle section 15 of the device body or close to the head section 14 of the device body and sensor 16 positioned at the tail section 18 of the device body. The body (shell) is preferably soft and flexible.

It should be appreciated that a different number of sensors (e.g., only one sensor or two or more sensors) can be provided and the sensors can be placed in locations other than those shown in FIG. 3. For example, an additional sensor can be placed at the head section 14, sensors can be placed on opposing sides of the device 10 or closer together or further apart from that shown. Thus, FIG. 3 shows one example of sensor placement. Further, the sensor(s) can be of different sizes and configurations than shown in FIG. 3.

The sensors are preferably built into the device and provide a smooth outer surface of the device (do not protrude radially with respect to the outer surface of the device) so as not to adversely affect patient comfort.

The sensors in some embodiments include power, measurement, calibration, and data communication functions compatible with load monitoring, force sensing for vaginal tissue interfaces, gait monitoring, athletic performance evaluation, excess pressure prevention or other pressure sensing application.

Various force or pressure sensors can be used such as uncompensated pressure sensors, temperature-compensated and calibrated sensors and integrated sensors.

The pressures sensors can provide an analog signal that is either processed by an analog-to digital converter or fed directly to a microcontroller which can provide data to a display and/or use the pressure signal and appropriate software to control the device.

Intelligent pressure sensing can also be provided which combines pressure sensors with microcontrollers and communications technology. Digital manipulation of real-time pressure signals can enable physicians and caregivers to monitor various parameters. Intelligent pressure sensing systems can allow physicians to remotely measure pressure and track patient compliance in a closed-loop system via the Internet.

Device 10 also includes a pair of electrodes supported on the device body, identified in FIGS. 3 and 5-7 as a left electrode 20 and a right electrode 22 in the orientation shown. The electrodes 20, 22 send an electrical signal to the pelvic muscle to recruit the muscle and contract the muscle. This is shown in FIGS. 8A and 8B. In FIG. 8A, the device 10 is “at rest” or “inactivated” in accordance with normal pressure in the abdomen, as represented by the broken arrow (blocks). When the electrodes 20, 22 apply electrical energy to the pelvic muscles in response to increased pressure, the muscles are contracted as shown in FIG. 8B to limit or stop leakage. The inwardly facing arrows in FIG. 8B illustrate the direction of pelvic muscle movement during electrical pulse-induced retraction. The downwardly pointed arrows illustrate the change in pressure with the smaller arrows of FIG. 8A illustrating normal pressure and the larger arrow of FIG. 8B indicating sudden increased pressure on the abdomen and bladder.

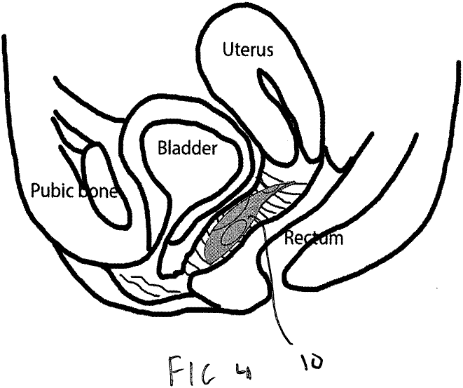

In the embodiment of FIGS. 3-7, the device 10 has two electrodes 20, 22 on opposing sides (right and left as viewed in the orientation of FIG. 5) of the device in middle section 15, i.e., between the head section 14 and tail section 18. The electrodes 20, 22 are also configured so that they extend radially only about a portion of the circumference of the device 10. It should be appreciated that a different number of electrodes (e.g., only one electrode or two or more electrodes) can be provided and placed in locations other than those shown in FIG. 5. For example, an additional electrode can be placed at the head 14 and/or tail 18 and/or in other regions of the device 10. Thus, FIG. 5 shows one example of electrode placement. Further, the electrode(s) can be of different sizes and configurations than that shown in FIG. 3.

The electrodes 20, 22 are preferably built into the device and provide a smooth outer surface of the device (do not protrude radially with respect to the outer surface of the device) so as not to adversely affect user comfort.

The device is relatively small (compact), lightweight and generally elliptical in shape as best shown in the anterior view of FIG. 5. This configuration best fits the natural vaginal shape. A small amount of lubricant can be provided to the device prior to insertion. The device is inserted with its tail section first. As explained herein, the device is also preferably ambulatory so the user can be mobile during use including bending, stooping and during exercise such as running.

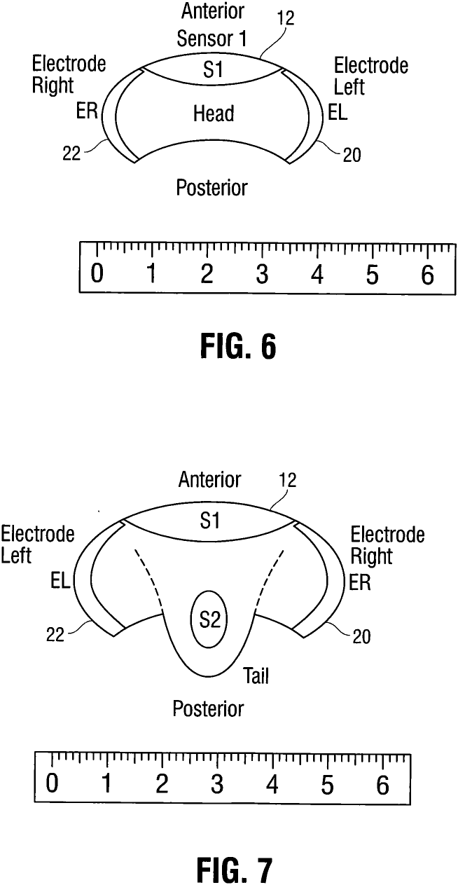

As shown, device 10 narrows at tail section 18 so that a transverse dimension T1 at the tail section 18 is less than a transverse dimension T2 at the middle section 15. The head section 14 also narrows to transverse dimension T3 greater than T1 and less than T2. Note the tail section 18 tapers in a distal direction toward a distalmost end so its narrowest dimension is at a distal end; the head section 14 reduces in cross-section in a proximal direction so its narrowest dimension is at the proximalmost end. The device 10 also has a curvature 19 (FIG. 3) transitioning from the middle section 15 to the tail section 18. Device 10 further has a concavity 17 (FIG. 6) spanning widthwise from the right to the left side of the device. In some embodiments, the device can include wings to further reduce the likelihood of accidental dislocation, e.g., the device falling out of the user’s body.

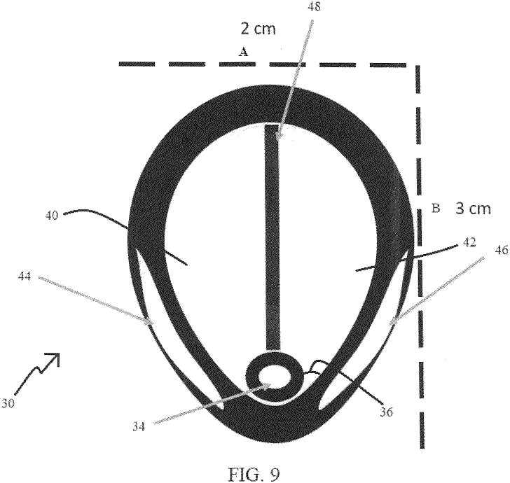

FIG. 9 illustrates an alternate embodiment of the stimulator device of the present invention. The device 30 has a different configuration than device 10 in that it is more ring-shaped and does not have the elongated tail. In the illustrated embodiment it has a width A of about 2 cm, a length B of about 3 cm and a diameter of about 1 cm. (Other dimensions are also contemplated). Device 30 has an extraction ring 34 closer to the vaginal opening which can be attached to the device body by a string 36 to facilitate removal from the vagina. Device 30 has two battery compartments - first battery compartment 40 and second battery compartment 42 to receive first and second batteries, respectively. In preferred embodiments, the compartments 40, 42 are openable to allow for replacement of the batteries. Since the device needs to be sufficiently flexible to fold to insert into the vagina and open up once in place to put the electrodes in contact with the side wall, the two compartments 40, 42 can provide more flexibility than a single compartment and battery in the center. The device 30 has side electrodes 44, 46 and top sensor 48 and a sensor on the opposite side. Note a different number and different location of the sensors and electrodes are contemplated. It should be understood that the various description of electrodes and sensors, remote devices, modes of operation, etc. discussed herein are fully applicable to the device 30 and for brevity are not repeated herein. It should also be understood that the extraction ring and double compartment can be used with device 10 of the embodiment of FIGS. 3-7.

The electrical signals are in preferred embodiments generated within the device 10 as the electric signal generator is housed within the body (shell). The device preferably is “all in one” and does not have external connectors. In some embodiments, the generator provides pulsed signals for providing electrical pulses to the muscles. The device can be powered by a battery received in the device for powering the electrodes and/or sensors. The battery can be rechargeable via a connection or magnetic recharge. The battery can be mounted within the deice so it is charged while mounted; alternatively, the battery is removable and can be removed from the device for charging. In some embodiments, the battery is replaceable with another battery.

In some embodiments, the user can manually increase or decrease the stimulation intensity that fits her needs best to control stress urinary incontinence. Thus, if an increased pressure is detected, the user can activate the device to transmit electric signals to the pelvic muscles to effect contraction. Based on the level of comfort of the stimulation, the user can adjust the level of intensity. Note the higher stimulation (intensity), the more intense contraction but the greater potential discomfort, so the user controls the comfort level. Thus, in these embodiments, rather than the user relying on a preset intensity level for electrical stimulation to the muscles, the user can set her own level, e.g., reduce the intensity if experiencing too high a discomfort level. Consequently, this embodiment not only provides “on demand” user control where user activation is necessary to apply the electric stimulation, but the intensity of the electrical energy can be modified at any time during use. The device itself (or an external device communicating with the device) can include a readout or another type of indicator to indicate and track the level of intensity to provide a baseline for further electrical pulses. The intensity levels can be numeric indicators or generalized indicators, e.g., high, medium, low, etc. Tables and graphical representations are also contemplated. Such indicators can be provided on remote devices for easy visualization or can be provided on the device itself for viewing after removal of the device. Data storage as described herein is also contemplated.

In an alternate embodiment, instead of user control, the sensors can detect increased abdominal/bladder pressure and automatically, without user intervention, transmit the signals via the electrodes to effect pelvic muscle contraction. In some embodiments, the user can initially preset the intensity, and in some embodiments can adjust the intensity to a new preset value at any time. User feedback can be used to set the intensity levels. The automatic electrical pulses can be tied into the degree of abdominal/bladder pressure. For example, in some embodiments, electrical stimulation can occur only if the pressure exceeds a predetermined threshold (parameter). In this manner, minor changes in pressure (below the preset threshold/parameter), e.g., due to slight movement or other reasons, would not trigger a response. The automatic electrical pulses can also be tied into comparative measurements, wherein stimulation is based on a predetermined degree of change.

In other alternate embodiments, the sensor can send a signal to the control to automatically activate the electrodes but the user can override the control or adjust the intensity of the automatic activation in preparation for the next activation. Readouts/indicators can also be provided in these embodiments, as well as storage of data for user and/or clinician analysis.

In the foregoing embodiments with user control, the device can be activated by a control outside the body via a wireless or Bluetooth connection. Such connection can enable the user to initially activate the device prior to insertion or alternatively activate the device once inserted. In some embodiments, the external control can include a graphic display (e.g., displayed on a monitor) to display the measured surge in abdominal pressure, the intensity of the electrical energy or other parameters such as time period. The external control in some embodiments can be configured to collect and store data of the time periods and/or intensity of the signals for later evaluation or for data collection for evaluation of data from multiple users. Thus, a microprocessor and memory can be provided in the external device in some embodiments. (In other embodiments, the device itself can include a microprocessor to process data generated by the sensor(s) and/or can include a memory to store data received from the sensor(s)). The sensors, as described herein, can be force sensors, pressure sensors, motion sensors (to detect patient and/or muscle movement), light sensors as well as any other sensors which can effectively activate the electrode(s) as described herein.

The device of the present invention can be operated in a “remote control mode” or a “computerized mode.” In the remote control mode, a Mobile App running on a smart phone provides via Bluetooth connectivity for biofeedback, electrical stimulation and pressured-triggered stimulation. In the computerized mode, software running on a personal computer provides for biofeedback and programming by the provider only.

The device can be operated via a graphical interface on an LCD screen and a capacitive touch pad with haptic and audio feedback. The connection to a phone, tablet or personal computer can be wireless via Bluetooth. Visual, audio and voice feedback, and prompts can be provided to facilitate device operation.

When used in biofeedback mode wherein electrical stimulation is tied into pressure measurements, the device can record stimulations and the intra-abdominal pressures. When used in a therapeutic mode, the standalone device can be used for pelvic floor electrical stimulation.

The device can be powered for example by an internal rechargeable Lithium-ion or lithium polymer battery via a magnetic charger for the Biofeedback mode (a more intermittent mode) to offer for example up to about 8 hours of device operation, or in Therapeutic mode (more continuous mode), between about 30 minutes to about 60 minutes, and preferably about 20 minutes, of continuous operation. Frequencies of about 20 HZ to about 80 Hz are contemplated, and in a continuous mode, preferably about 20 HZ. In the reconnaissance mode, it can be activated only in response to elevated intra-abdominal pressures.

Various energy application parameters are contemplated. In one embodiment by way of example, the current is about between about 56 and about 74 mA with a maximum current of 74 mA +/- 18% at 500 ohm. Duration of each stimulation can be between about 0.2 mins. and about 0.5 mins. and the working time (intermittent) can be about 12 hours while continuous working time can be about 60 minutes before recharging.

AI based learning is also contemplated for sensing and energy application.

Note that motion, ultrasound, light detection or other methods can be used to communicate with and activate the electrode(s) to train pelvic floor muscles and/or treat urinary incontinence, fecal incontinence, pelvic organ prolapse, etc.

The sensors can also be configured/set so that electrical stimulation is not provided, or the user is not alerted, if the pressure does not exceed a threshold value. Thus, in these embodiments, the sensor would read pressure and send a signal for electrical energy, e.g., electrical pulse generation, only if the abdominal pressure was determined to exceed a preset level. If the level is exceeded, then the signal would trigger activation in the automatic stimulation embodiments or alert the user via a display or other visual or audible alert in the on-demand embodiments. In some embodiments, the threshold (preset) vaginal pressure could be 100 mm Hg so pressure below this amount would not trigger the stimulator and pressure above this amount would trigger the sensors. Other pressure thresholds are also contemplated.

In some embodiments, the electrodes can provide by way of example, an impulse of Biphasic current, frequency between about 30 HZ and about 50 HZ, and preferably about 30 HZ, a pulse width about .25 ms, and a current intensity of between about 30 mA to about 50 mA, and preferably about 30 mA, and preferably with no off-on cycle to the side electrodes. Other frequencies and currents are also contemplated.

In some embodiments, the stimulator device is connected to an electronic device such as a tablet, iPad, smart phone, etc. so the intensity is controlled via the electronic device which can also capture the pressure and motion gradient. Thus, the smart phone capability eliminates the need to dial up from the device itself. In such embodiments, the electronic device can also be used to collect data regarding the sensing of pressure, the intensity and frequency of electrical stimulation and other parameters for customization to a patient and/or for data collection for research and analysis to assess efficacy and control and stimulation based on a number of users.

The present invention is also directed to various methods of use of the device disclosed in the Figures and described above for use in muscle training and/or for use in treatment of urinary incontinence, fecal incontinence and/or pelvic floor weakness. For example in one method, the method of use includes remotely activating the device via an external control in wireless communication with the device, wherein activating comprises either i) enabling a selective mode wherein the user remotely selectively sends a signal to the electrode to send electrical energy to contract the pelvic muscles in response to measured intra-abdominal pressure by the sensor; or ii) enabling an automatic mode wherein the signal is automatically sent to the electrode to send electrical energy to contract the pelvic muscles in response to measured intra-abdominal pressure by the sensor.

In another method, the method of use includes using the device when the user is stationary or in an ambulatory/active position/condition.

In another method of use, the method includes collecting data during use of pressure measurement, electrical energy application, e.g., intensity, abdominal pressure measurements, etc., and the data can be stored in memory in the device and in addition, or in lieu of, stored in memory on a remote device.

Thus, the methods of use can be implemented in accordance with the different modes of operation described herein.

It should be understood that the foregoing description of device functions, communication/operation with remote devices, clinical uses, operation modes, methods of use, etc. are applicable to stimulator device 10 of FIGS. 3-7 and to stimulator device 30 of FIG. 9.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

Where a range of values is provided, it is understood that each intervening value, between the upper and lower limit of that range is encompassed within the invention.

Although the apparatus and methods of the subject invention have been described with respect to preferred embodiments, which constitute non-limiting examples, those skilled in the art will readily appreciate that changes and modifications may be made thereto without departing from the spirit and scope of the present invention as defined by the appended claims.

It will be understood by those skilled in the art that the above particular embodiments are shown and described by way of illustration only. The principles and the features of the present disclosure may be employed in various and numerous embodiments thereof without departing from the scope and spirit of the disclosure as claimed. The above-described embodiments do not restrict the scope of the disclosure.

Additionally, persons skilled in the art will understand that the elements and features shown or described in connection with one embodiment may be combined with those of another embodiment without departing from the scope of the present invention.

Throughout the present disclosure, terms such as “approximately,” “about,” “generally,” “substantially,” and the like should be understood to allow for variations in any numerical range or concept with which they are associated. For example, it is intended that the use of terms such as “approximately,” “about” and “generally” should be understood to encompass variations on the order of 25%, or to allow for manufacturing tolerances and/or deviations in design.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. An intravaginal stimulator device comprising:
a) a body configured for insertion intravaginally for placement within the vagina, the body having a head section, a middle section and a tail section;
b) at least one sensor supported on the body, the sensor measuring intra-abdominal pressure;
c) at least one electrode supported on the body and in electrical communication with the at least one sensor, the at least one electrode sending an electrical signal to pelvic muscles of a user to contract the pelvic muscles based on feedback from the sensor; and
d) an electric signal generator housed within the body for sending the electrical signal to the electrode;
e) wherein the device is configured for remote activation by an external control in wireless communication with the device, wherein the device has a selective mode and an automatic mode, in the selective mode a signal is sent by the generator to the at least one electrode to send electrical energy in response to measured abdominal pressure based on a selective activation by the user to contract the pelvic muscles and in the automatic mode the signal is automatically sent to the electrode to send electrical energy to contract the pelvic muscles in response to measured intra-abdominal pressure by the sensor.

2. The device of claim 1, wherein the intensity of electrical stimulation is adjustable by the user.

3. The device of claim 1, wherein the at least one sensor comprises a first sensor at the tail section and a second sensor at the head section.

4. The device of claim 1, wherein the at least one sensor comprises a first sensor on one side of the body and a second sensor on an opposing side of the body.

5. The device of claim 1, wherein the at least one electrode is built into the body to provide a smooth outer surface of the device.

6. The device of claim 1, wherein the electrical signals are generated within the device by the generator within the body such that the device does not have an external connector and is fully contained within the vagina.

7. The device of claim 1, wherein a signal is sent to the electrode only if intra-abdominal pressure exceeds a predetermined threshold.

8. The device of claim 1, wherein a signal is sent to the electrode only if an increase in intra-abdominal pressure exceeds a predetermined amount.

9. The device of claim 1, wherein one or both of light detection or motion detection activates the at least one electrode.

10. The device of claim 1, wherein the external control comprises a smart external electronic control, the external control having a graphic display to display one or more of a) abdominal pressure; b) a surge in abdominal pressure; and/or c) intensity of the electrical energy.

11. The device of claim 1, wherein data is collected and stored of parameters of use of the device of one or more of a) abdominal pressure; b) a surge in abdominal pressure; c) intensity of the electrical energy; and/or d) time period of electrical energy application, the data stored on one or both of the device and the external control.

12. The device of claim 1, wherein the external control is a smart phone and the device is configured to be in communication with a mobile app running on the smart phone which provides for biofeedback and electrical stimulation.

13. The device of claim 1, wherein the external control is a personal computer and the device is configured to be in communication with software running on the personal computer to provide for biofeedback and programming by a provider only.

14. The device of claim 1, wherein the device is configured for use during a stationary and an ambulatory condition of the user.

15. An intravaginal stimulator device comprising:
a) a body configured for insertion intravaginally for placement within the vagina, the body having a head section, a middle section, and a tail section, the tail section having a transverse dimension less than a transversion dimension of the middle section;
b) at least one sensor supported on the body, the sensor measuring intra-abdominal pressure;
c) at least one electrode supported on the body and in electrical communication with the at least one sensor, the at least one electrode sending an electrical signal to pelvic muscles of a user to contract the pelvic muscles based on feedback from the sensor; and
d) an electric signal generator housed within the body for sending the electrical signal to the electrode.

16. The device of claim 15, wherein the device is configured for use during a stationary and an ambulatory condition of the user.

17. A method for contracting pelvic muscles comprising:
a) intravaginally inserting a device having i) at least one sensor supported on the body for measuring intra-abdominal pressure, ii) at least one electrode supported on the body and in electrical communication with the at least one sensor, and iii) an electric signal generator housed within the body for sending the electrical signal to the electrode the at least one electrode to send electrical energy to pelvic muscles of a user to contract the pelvic muscles based on feedback from the sensor; and
b) remotely activating the device via an external control in wireless communication with the device, wherein activating comprises either i) enabling a selective mode wherein the user remotely selectively sends an electrical signal to the at least one electrode to send electrical energy to the pelvic muscles to contract the pelvic muscles in response to measured intra-abdominal pressure by the sensor; or ii) enabling an automatic mode wherein the electrical signal is automatically sent to the at least one electrode to send electrical energy to the pelvic muscles to contract the pelvic muscles in response to measured intra-abdominal pressure by the sensor.

18. The method of claim 17, wherein the device is usable in stationary and active positions of the user.

19. The method of claim 17, wherein the device records electrical stimulation and measured intra-abdominal pressures.

20. The method of claim 17, wherein the intensity of electrical stimulation is adjustable by the user.
